SEQUENCE LISTING (16 sequences)

SEQ ID NO: 1 - protein, 330 aa, Suberites domuncula
MLVTVVVLGLLGFASAAQPKFEFVEEWQLWKSTHSKMYESQLMELERHLTWLSNKKYIEQHNVNSHIFGFTLAMNQFGDLSELEYANYLGQYRIEDKKSGNYSKTFQRDPLQDYPEAVDWRTKGAVTAVKDQGDCGASYAFSAMGALEGANALAKGNAVSLSEQNIIDCSIPYGNHGCHGGNMYDAFLYVIANEGVDQDSAYPFVGKQSSCNYNSKYKGTSMSGMVSIKSGSESDLQAAVSNVGPVSVAIDGANSAFRFYYSGVYDSSRCSSSSLNHAMVVTGYGSYNGKKYWLAKNSWGTNWGNSGYVMMARNKYNQCGIATDASYPTL

SEQ ID NO: 2 - protein, 191 aa, Mytilus galloprovincialis
GGLGGAGPGGLGGLGGGLGGLGGGLGGLGGAGGLGGGLGGLGGAGGLGGGLGGLGGAGGGAGGAGGAGAIAAAAAQAAAAAGAGGNGGAAQAAAQAAASAAANSGLGAGAARAAASAAARATVAGHGSGTAAAAANAAAQAHAATRGQGGSHAHAAAAAHAAASSVIHGGGHGGHGGDYHGGDAGYHKPGY

SEQ ID NO: 3 - DNA, 990 bp, Suberites domuncula
atgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccacccta

SEQ ID NO: 4 - DNA, 573 bp, Mytilus galloprovincialis
ggtggactcggaggagcaggaccaggaggactcggaggactaggaggtggactcggaggacttggaggtggactcggaggattaggaggagcaggaggtttaggtggtggactcggaggattaggaggagcaggaggtttaggtggtggactcggaggattaggaggagcaggaggtggagcaggtggagcaggaggagcaggtgcaatagcagctgcagcagcacaagcagcagcagcagcaggagcaggaggaaacggtggagcagcacaagcagcagcacaagcagcagcatcagcagcagcaaattcaggacttggagcaggagcagcaagagcagcagcatcagcagctgctagagcaaccgtagcaggacatggaagtggaaccgccgcagcagcagccaacgcagccgcacaagcacatgcagcaacacgaggacaaggaggatcacacgcacacgctgccgccgcagctcacgcagccgcaagtagcgtaatccatggtggtggacacggtggacatggtggtgactatcacggaggcgatgccggctatcacaaaccaggatat

SEQ ID NO: 5 - DNA, 1200 bp, Mytilus galloprovincialis
gaggatagaaagtctacaatctgtaaggacaatgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccaccctataaacttatatatatatagtcttagaaacattatccttttctttacccttgtctctataggccatagagtgattgtaggctgtttgcatttgatgactgtatataccctatcattttttgtgattctatctgattaaaaatcccatacccgaccaaaccatcaatttatcaaatcatga

SEQ ID NO: 6 - DNA, 2848 bp, Mytilus galloprovincialis
gtggtatggtctacaaactcctgaccgtgtgtcttgtagcatctcttctagagatttgcttagctgactatcacggtaacaaacagtatggcggcagatacggcaacagattcggaggtggtattggtggtgcaggagccgtagcccatgcccatgcccatgcccatgctagtgccggagcaaacggaagagcaagagcacatgcacgagccttggcccatggacatgccggtggtagcgctgcacatggacacccaggattcccaattggtggtagcgcaagcgcagccgcacgtgcagcagcacgagcatcagcaggaggattaggaggactaggtggattcgtatcagcagcagccaatgcagcagcagcagcaaacgcaggagcaggatttggtggattcggtggattcggaggactcggaggagtcggcggtccaggtcaaccaggcggtccaggaggtccaggaggtccaggaggtccaggcggtccaggtatgccaggcggtccaggaggtccatcaggtccaggtaccggaggtccaggtcaaccagatggtccaggaggtccaggcggtccaggaggtccaggtggtccaggtatgccaggcggtccaggaggtccaggtggaccaggaggaccaggaagaccaggtgttggaggaggtgatggaggaatcccaggcatgcccggaccagcaggaccaccaggcccagccggcccacaaggaccagaaggcgaacaaggaccacgaggacgaaccccagcaggaacaccaggcccaccaggcaatccaggagaaccaggacaagcaggagcaccaggcgcaccaggagcaccaggacatgccggtaaacacggaaccgcaggaccagcaggcaaagcaggacgtccaggaccaagaggacaatcaggagcatcaggatcatcaggccaacacggagcatcaggagcaccaggtcgtccaggtaacccaggatccaccggacgtccaggagcaaccggagacccaggtcgtccaggagcaaccggaacaacaggaagaccaggaccatcaggagcaccaggaaacccaggagcaccaggagcactcggagcaccaggaccacgaggagcaccaggatttgtcggacttccaggaccacgaggatccccaggagaaccaggaaaccaaggaccaatcggaggcccaggatacccaggaccacgaggaccacaaggacctgatggtgctatgggaccacaaggaccatgtggagatagaggagcaccaggagtaccaggcaaacaaggaccagtaggaggacaaggaccagcaggaccacgaggaccacgaggagatggaggaccagttggaccaaagggcgaaccaggagcaaaaggagctgatggtaaaccaggagacagaggacctgatggagaaaccggaccacaaggaccagctggaccaaagggacaagtaggagaccaaggcaaaccaggagcaaagggagaaaccggagatcaaggagcacgaggtgaagcaggaaaggccggagaacaaggaccaggaggcatccaaggaccaaagggaccagtaggaggacaaggaccagcaggaccaagaggaccacgaggagatgaaggaccagttggaccaaagggcgaaccaggagcaaagggagctgatggtaaaccaggagacagaggacctgatggagaaaccggaccacaaggaccagctggaccaaagggacaagtaggagaccaaggagcacaaggagaccaaggagcaagtggcgctgatggcaaaaagggagaaccacgagagagaggacaacaaggagcagcaggaccagtcggccgaccaggaccaagaggagatagaggagcaaagggaatccaaggaagccgaggacgaccaggtggtatgggtagacgaggaaaccgaggatcccaaggagcagtaggaccacgaggagaaactggcccagacggtaaccaaggacaacgtggagaacaaggagcaccaggagttatcacccttgtcattgaagacctcagaacagccggagtagaaagccccgtagaaacatttgacgcaggagcaggaaccggtggaccagcaccaggagcaggagcaacagcaggagcatttgcaggagcaggaccaggaggagctaatgcaggaggaaacgcagccgcaggagcaggaccaggtggactcggaggagcaggaccaggaggactcggaggactaggaggtggactcggaggacttggaggtggactcggaggattaggaggagcaggaggtttaggtggtggactcggaggattaggaggagcaggaggtttaggtggtggactcggaggattaggaggagcaggaggtggagcaggtggagcaggaggagcaggtgcaatagcagctgcagcagcacaagcagcagcagcagcaggagcaggaggaaacggtggagcagcacaagcagcagcacaagcagcagcatcagcagcagcaaattcaggacttggagcaggagcagcaagagcagcagcatcagcagctgctagagcaaccgtagcaggacatggaagtggaaccgccgcagcagcagccaacgcagccgcacaagcacatgcagcaacacgaggacaaggaggatcacacgcacacgctgccgccgcagctcacgcagccgcaagtagcgtaatccatggtggtggacacggtggacatggtggtgactatcacggaggcgatgccggctatcacaaaccaggatattaagctaattgacaataatagacattaaaatattgtaaattgaatatgttactttatcaataaaatctgcaagtgtt

SEQ ID NO: 7 - protein, 922 aa, Mytilus galloprovincialis
MVYKLLTVCLVASLLEICLADYHGNKQYGGRYGNRFGGGIGGAGAVAHAHAHAHASAGANGRARAHARALAHGHAGGSAAHGHPGFPIGGSASAAARAAARASAGGLGGLGGFVSAAANAAAAANAGAGFGGFGGFGGLGGVGGPGQPGGPGGPGGPGGPGGPGMPGGPGGPSGPGTGGPGQPDGPGGPGGPGGPGGPGMPGGPGGPGGPGGPGRPGVGGGDGGIPGMPGPAGPPGPAGPQGPEGEQGPRGRTPAGTPGPPGNPGEPGQAGAPGAPGAPGHAGKHGTAGPAGKAGRPGPRGQSGASGSSGQHGASGAPGRPGNPGSTGRPGATGDPGRPGATGTTGRPGPSGAPGNPGAPGALGAPGPRGAPGFVGLPGPRGSPGEPGNQGPIGGPGYPGPRGPQGPDGAMGPQGPCGDRGAPGVPGKQGPVGGQGPAGPRGPRGDGGPVGPKGEPGAKGADGKPGDRGPDGETGPQGPAGPKGQVGDQGKPGAKGETGDQGARGEAGKAGEQGPGGIQGPKGPVGGQGPAGPRGPRGDEGPVGPKGEPGAKGADGKPGDRGPDGETGPQGPAGPKGQVGDQGAQGDQGASGADGKKGEPRERGQQGAAGPVGRPGPRGDRGAKGIQGSRGRPGGMGRRGNRGSQGAVGPRGETGPDGNQGQRGEQGAPGVITLVIEDLRTAGVESPVETFDAGAGTGGPAPGAGATAGAFAGAGPGGANAGGNAAAGAGPGGLGGAGPGGLGGLGGGLGGLGGGLGGLGGAGGLGGGLGGLGGAGGLGGGLGGLGGAGGGAGGAGGAGAIAAAAAQAAAAAGAGGNGGAAQAAAQAAASAAANSGLGAGAARAAASAAARATVAGHGSGTAAAAANAAAQAHAATRGQGGSHAHAAAAAHAAASSVIHGGGHGGHGGDYHGGDAGYHKPGY

SEQ ID NO: 8 - protein, 191 aa, Mytilus galloprovincialis; identical to SEQ ID NO: 2

SEQ ID NO: 9 - DNA, 573 bp, Mytilus galloprovincialis; identical to SEQ ID NO: 4

SEQ ID NO: 10 - DNA, 29 bp, Suberites domuncula
ccatggttcttgtcacagtggtagtactg

SEQ ID NO: 11 - DNA, 28 bp, Suberites domuncula
ccatggatagggtgggataagatgcatc

SEQ ID NO: 12 - DNA, 17 bp, Mytilus galloprovincialis
ggtggactcggaggagc

SEQ ID NO: 13 - DNA, 20 bp, Mytilus galloprovincialis
atatcctggtttgtgatagc

SEQ ID NO: 14 - DNA, 29 bp, Suberites domuncula
cttgtcatcgtcatctagggtgggataag

SEQ ID NO: 15 - DNA, 29 bp, Suberites domuncula
ccatggttcttgtcacagtggtagtactg

SEQ ID NO: 16 - DNA, 26 bp, Suberites domuncula
ccatggacttgtcatcgtcatctagg

(19) United States
(12) Patent Application Publication
(10) Pub. No.: US 2010/0047224 A1
(43) Pub. Date: Feb. 25, 2010

(54) Biosilica-Adhesive Protein Nanocomposite Materials: Synthesis and Application in Dentistry

(76) Inventors: Werner K. Geurtsen, Wedemark (DE); Werner E.G. Müller, Wiesbaden (DE); Heinz C. Schröder, Wiesbaden (DE)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/438,441

(22) PCT Filed: Aug. 21, 2007
(86) PCT No.: PCT/EP07/07363

§ 371 Date: Jul. 17, 2009

Related U.S. Application Data
(60) Provisional application No. 60/839,601, filed on Aug. 23, 2006.

(51) Int. Cl. A61K 38/44; C12N 9/02; C07H 21/04; C12P 9/00; C12P 21/00; C12N 15/63; C12N 5/10; A61P 31/04

(52) U.S. Cl. 424/94.3; 435/188; 536/23.2; 435/131; 435/68.1; 435/320.1; 435/325

(57) ABSTRACT

The invention concerns the application of silicatein-silk fibroin fusion proteins in dentistry to synthesize silica-containing nanocomposite materials used as filling material.

### BACKGROUND OF INVENTION

Silica is widely used in industry and medicine, e.g. for the fabrication of glasses, ceramics, paints, adhesives, and catalysts, as component of molecular sieves, as food additive, carrier, stabilizer (e.g. in toothpaste), and as an insulator in semiconductor devices. Silica is also an important material in nano(bio)technology. The technological production of silica mostly requires high temperature conditions and extremes of pH. Noteworthy, certain single- and multi-cellular organisms, including diatoms, sponges and higher plants are able to form their silica skeletons under ambient, low temperature and pressure and near-neutral pH conditions. In addition, the skeletal elements of these organisms are produced with high fidelity and in large copy number, making these organisms and the mechanism(s) underlying the formation of their skeletons of interest for the fabrication of novel biosilicas with unique properties.

Marine and freshwater sponges have the unique ability to synthesize silica (biosilica) enzymatically. This ability makes sponges highly interesting for (nano)biotechnology. Hitherto used methods for the production of silica (glass) require the presence of high temperature, pressure and aggressive chemicals. Sponges are able to synthesize silica nanostructures by enzymes (biocatalysts) under biological and environmentally benign conditions with great precision and reproducibility.

The main elements of the skeleton of siliceous sponges are the needle-like spicules, which consists in the class of Demospongia and Hexactinellida of amorphous non-crystalline silica.

The state of art in morphology and biogenesis of spicules is described in: Uriz et al. (2003) Progr Molec Subcell Biol 33:163-193; Müller et al. (2003) Progr Molec Subcell Biol 33:195-221. The opal silica of sponge spicules contains 6-13% water, corresponding to the formula (SiO₂)₂₋₅.H₂O (Schwab & Shore (1971) Nature 232:501-502). Spicule formation in demosponges starts around an axial filament, around which the silica is deposited enzymatically.

Two enzymes and their technical application have been described, which are involved in synthesis and/or degradation of the SiO₂ skeleton in silica forming organisms.

The first enzyme is silicatein-a (also termed silicatein) which exist in the axial filament of the sponge spicules (needles) (PCT/US99/30601. Methods, compositions, and biomimetic catalysts, such as silicateins and block copolypeptides, used to catalyze and spatially direct the polycondensation of silicon alkoxides, metal alkoxides, and their organic conjugates to make silica, polysiloxanes, polymetallo-oxanes, and mixed poly(silicon/metallo)oxane materials under environmentally benign conditions. Inventors/Applicants: Morse D E, Stucky G D, Deming, T D, Cha J, Shimizu K, Zhou Y; DE 10037270 A 1. Silicatein-vermittelte Synthese von amorphen Silicaten und Siloxanen und ihre Verwendung. Inventors/Applicants: Müller W E G, Lorenz B, Krasko A, Schröder H C; European Patent No. 1320624. Silicatein-mediated synthesis of amorphous silicates and siloxanes and use thereof. Inventors/Applicants: Müller W E G, Lorenz B, Krasko A, Schröder H C; national phases: US2003134391; NO20030407; Japan No. 2002-516336; CN1460110T; Canada No. 2,414,602; Australia No. 2001289713). This enzyme has been cloned from the marine siliceous sponge Suberites domuncula (Krasko et al. (2000) Eur J Biochem 267:4878-4887). Silicatein is able to synthesize amorphous silica (polysilicic acid, polysilicate) from organic silicon compounds (alkoxysilanes) (Cha et al. (1999) Proc Natl Acad Sci USA 96:361-365).

Besides silicatein-α, further silicateins were cloned by the inventors, including silicatein-β (patent application: DE10352433.9. Enzym-und Template-gesteuerte Synthese von Silica aus nicht-organischen Siliciumverbindungen sowie Aminosilanen und Silazanen und Verwendung. Applicant: University of Mainz. Inventors: Schwertner H, Müller W E G, Schröder H C) and four silicatein isoforms from a freshwater sponge (silicatein-a1-4; DE102006001759.5. Kontrollierte Herstellung von Silber-und Gold-Nanopartikeln und Nanokristallen definierter Gröβe und Form durch chirale Induktion mittels Silicatein. Applicants/Inventors: Tremel W, Tahir M N, Müller W E G. Schröder H C). Silicatein retains its catalytic activity after binding to surfaces (Tahir et al. (2004) Chem Commun 2004:2848-2849).

The second enzyme is the silicase which belongs to the group of carbonic anhydrases (German Patent DE10246186. In vitro and in vivo degradation or synthesis of silicon dioxide and silicones, useful e.g. for treating silicosis or to prepare prosthetic materials, using a new silicase enzyme. Applicant: University of Mainz. Inventors: Müller W E G, Krasko A, Schröder H C; PCT/EP03/10983. Abbau und Modifizierung von Silicaten und Siliconen durch Silicase und Verwendung des reversiblen Enzyms. Applicant: University of Mainz. Inventors: Müller W E G, Krasko A, Schröder H C). This enzyme, which has first been discovered in the marine sponge S. domuncula, is able to dissolve silica under formation of free silicic acid (Schröder et al. (2003) Progr Molec Subcell Biol 33:250-268). In addition, the silicase—in the reversible reaction—may also mediate the synthesis of the polymer (DE10352433.9. Enzymatische Synthese, Modifikation und Abbau von Silicium(IV)—und anderer Metall(IV)—Verbindungen. Applicant: University of Mainz. Inventors: Müller W E G, Schwertner H, Schröder H C). Like silicatein, the silicase is of interest for nanotechnology, e.g. for modification of silica matrices in medicine and microelectronics.

Silica is an important component of materials used as a scaffold in tissue engineering bone and cartilage, including bioactive glasses and composite materials (Hench and Wilson (1984) Science 226:630-636; Yamamuro et al. (1990) Handbook on Bioactive Ceramics, Vol I: Bioactive Glasses and Glass-Ceramics, CRC Press, Boca Raton, Fla.). Biocompatibility and stability are critical features that determine the applicability of these materials, and there is an increasing demand to improve these materials for their use in surgery (e.g. for bone replacement) and dentistry. The chemical synthesis of polymeric silica and other siloxane-based materials typically requires drastic conditions such as high temperatures and high pressures, and the use of caustic chemicals, which may damage organic molecules used as components of composite materials. However, siliceous sponges are able to synthesize their silica skeleton under ambient (low temperature and pressure) conditions, making use of the biocatalytic activity of silicateins.

The present inventors showed that mineralization (formation of calcium phosphate) of human osteosarcoma SaOS-2 cells is markedly increased when grown on culture plates precoated with silicatein and type 1 collagen, and subsequently modified by coating with biosilica, using the silicatein substrate, TEOS (Schröder et al. (2005) J Biomed Mater Res Part B: Appl Biomater 75B:387-392; DE102004021229.5. Enzymatisches Verfahren zur Herstellung bioaktiver, Osteoblasten-stimulierender Oberflächen und Verwendung. Applicant: University of Mainz. Inventors: Schwertner H, Müller W E G, Schröder H C). The results show that biosilica-modified surfaces are bioactive and may be used to enhance osteoblast function.

The availability of the recombinant silica-synthesizing enzyme (silicatein) opens new ways for the biosynthesis of silica-containing bioactive surfaces under mild conditions that do not damage biomolecules. The inventors have also worked out the technology to produce the new biomaterial, sponge biosilica, in a sustainable way: this was achieved by establishing the sponge primmorph culture (a special form of sponge cell culture; patent/patent application: DE19824384.7, PCT/EP99/03121, EP99955288.8). Silica production of primmorphs can be increase by certain additives (EP05012162.3. Selenium-enriched liquid media for the cultivation of siliceous sponges and for biogenic silica production. Applicant: University of Mainz. Inventors: Müller W E G, Schröder H C, Osinga R, Schwertner H).

### Adhesive Proteins

Adhesive proteins are known from mussels (mussel adhesive proteins, MAPs, e.g. foot protein 1, Mefp-1). Mussels are able to attach themselves via adhesive plaques, which are composed of adhesive proteins, to metal, ceramics and glass surfaces. These adhesive proteins contain a high percentage of 3,4-dihydroxy phenylalanine (DOPA). Mefp-1 from Mytilus edulis has a tandem-like repeating decapeptide with the sequence Ala-Lys-Pro-Ser-Tyr-DHP-Hyp-Thr-DOPA-Lys. The adhesion to surfaces is mediated by the catechol oxygens.

Adhesive proteins also exist in other marine organisms, e.g. Holothuria. The inventors have been studied and described for the first time the biochemical adhesive mechanism of the Cuvier organs of Holothuria, Holothuria forscåli (Müller et al. (1972) Cytobiologie 5:335; Müller et al. (1976) Biochim Biophys Acta 433:684); FIG. 1. In addition, the inventors have shown that also sponges contain a tyrosinase (Müller et al. (2004) Micron 35:87) which converts monophenols in diphenols (FIG. 2).

### SUMMARY OF INVENTION

The invention relates to methods and the use of recombinant silicatein-silk fibroin fusion proteins for synthesis of amorphous silicon dioxide (silica), siloxanes and modifications of these compounds and the medical use thereof in dentistry.

### DESCRIPTION OF INVENTION

A fusion protein comprising a silicatein (silica forming sequence) sequence and a silk fibroin sequence and a cDNA coding for such a fusion protein are described. Silicatein is used for biocatalytic formation of (bio)silica that can serve as dental filling material either alone or as a component of a nanocomposite. Silk fibroin is used as an adhesive protein (“underwater glue”) to attach silicatein and silica nanoparticles formed by silicatein to enamel of teeth or surfaces of other solid materials including metals, plastics and composites. Several types and isoforms of silicatein can be used to construct the fusion protein, for example a cDNA coding for a silicatein-α polypeptide isolated from S. domuncula using the PCR method. Related cDNAs can be isolated from other marine sponges, e.g. Geodia cyclonium, or from freshwater sponges, e.g. Lubomirskia baicalensis. The fusion proteins, or parts of it, can be combined with human enamel-derived peptides or DOPA-containing peptides/proteins allowing the design of silica/peptide-based nanocomposites.

A further aspect of the invention is a procedure for in vitro or in vivo synthesis of silica (condensation product of silicic acid and/or silicate), silicones and other metal oxides as well as mixed polymers of these compounds, wherein a fusion protein is used, comprising a silicatein-α-domain which exhibits at least 25% sequence homology, preferably identity, to the sequence shown in SEQ ID No. 1.

For synthesis, a procedure is used, wherein compounds such as silicic acid, monoalkoxysilanetriols, monoalkoxysilanediols, monoalkoxysilanols, dialkoxysilane-diols, dialkoxysilanols, trialkoxysilanols, tetraalkoxysilanes, alkyl-, aryl- or metallo-silanetriols, alkyl-, aryl- or metallo-silanediols, alkyl-, aryl- or metallo-silanols, alkyl-, aryl- or metallo-monoalkoxysilanediols, alkyl-, aryl- or metallo-monoalkoxysilanols, alkyl-, aryl- or metallo-dialkoxysilanols, alkyl-, aryl- or metallo-trialkoxysilanes can be used as substrates. By using defined mixtures of these compounds, mixed polymers can be produced.

According to a further preferred aspect of the invention, defined 2- and 3-dimensional structures can be formed by binding of the fusion protein to other molecules or the surfaces of glass, metals, metal oxides, plastics, biopolymers or other materials as a template.

According to a further preferred aspect of the invention, a procedure for modification of hydroxyapatite (example: enamel), silica or metal oxide containing structures or surfaces is presented, wherein a fusion protein is used for modification, which comprises a silicatein and/or silk fibroin domain, which has at least 25% sequence homology, preferably identity, to the sequences shown in SEQ ID No. 1 and SEQ ID No. 2.

A further preferred aspect of the invention concerns a chemical compound or silica-containing structure or surface which has been obtained by using the procedure described herein.

A further preferred aspect of the invention concerns a fusion protein of silicatein-α from S. domuncula according to SEQ ID No. 1 or a homologous polypeptide which has in the amino acid sequence of the silicatein-α domain at least 25% sequence homology, preferably identity, to the sequence shown in SEQ ID No. 1 or parts thereof.

“Homology” is defined as the percentage of residues in a candidate amino acid sequence that is identical with the residues in the reference sequence silicatein-α domain after aligning the two sequences and introducing gaps, if necessary, to achieve the maximum percent homology. Methods and computer programs for the alignment are well know in the art. One computer program which may be used or adapted for purposes of determining whether a candidate sequence falls within this definition is “Align 2”, authored by Genentech, Inc.

A further preferred aspect of the invention concerns a nucleic acid, in particular according to SEQ ID No. 3 and SEQ ID No. 4, wherein this nucleic acid codes for a polypeptide described in this patent application. The nucleic acid can be a DNA, cDNA, RNA or a mixture thereof. The sequence of the nucleic acid can comprise at least one intron and/or a polyA sequence.

A further preferred aspect of the invention concerns a nucleic acid in form of a (a) fusion protein (chimeric protein) construct or (b) construct with separate protein expression (protease cleavage site). The nucleic acid can also be produced synthetically. The necessary methods are state of the art.

A further preferred aspect of the invention concerns a vector, preferentially in form of a plasmid, shuttle vector, phagemid, cosmid, expression vector, retroviral vector, adenoviral vector or particle, nanoparticle or liposome, wherein the vector contains a nucleic acid according to the invention. Furthermore, these vectors can be used for the transfer of proteins, preferentially in form of nanoparticles or liposomes, comprising a fusion protein according to the invention.

A further preferred aspect of the invention is a host cell transfected with a vector or infected or transduced with a particle according to the invention. This host cell can express a polypeptide according to claims 1 to 7 or parts thereof. Any known host cell organism such as yeast, fungi, sponges, bacteria, CHO cells or insect cells can be used.

The fusion protein claimed herein can be produced synthetically or be present in a prokaryotic or eukaryotic cell extract or lysate. The cell extract or lysate can be prepared from a cell ex vivo or ex vitro, for example from a recombinant bacterial cell.

The fusion protein claimed herein can be purified using state-of-the-art methods and can therefore be essentially free of other proteins.

The adhesive protein present in the fusion protein can be used for the controlled attachment of silicatein and biosilica building blocks to surfaces.

Furthermore, the fusion protein or the nucleic acid according to the invention can also be used to modulate the metabolism and resorption of silicones and silicone implants. Silicatein is able to synthesize polymeric silicones from monomeric silicone precursors, and can thus be used for the removal of monomers which can be taken up by body cells. Finally, the invention described herein can be applied for transfection of cells with the nucleic acids described herein for modulating the metabolism and resorption of silicones and silicon implants. The methodologies for the above-mentioned applications are state-of-the-art and can easily be adapted to the specific requirements.

Moreover, diphenol-containing proteins/peptides, in particular DOPA (3,4-dihydroxy phenylalanine)-containing proteins/peptides are known to serve as glue in aqueous environments. The inventors cloned a sponge tyrosinase and express the recombinant protein. This enzyme synthesizes diphenols using monophenol compounds. The DOPA-containing proteins and peptides prepared by using the recombinant sponge enzyme can be used to bind silicatein/biosilica building blocks on surfaces.

Through adhesion of specifically modified DOPA-containing proteins and peptides, patterns on surfaces of different materials (metal, glass, plastic etc.) by coating of the surfaces with DOPA-containing proteins and peptides can be generated.

Both metal surfaces (titanium, titanium alloys or CoCr) and surfaces from selected plastic and composite materials can be used. The catechol oxygens are important for the binding of the DOPA-containing peptides and proteins on surfaces, which possess strong binding affinities in particular to Fe(III) and other metal ions.

The production of a stable connection between bone and implants (e.g. metal implants made of Ti, Ti-alloys or CoCr) is a major problem. One way for “biologisation” of metal implants is a coating of the surface with DOPA-containing proteins and peptides.

### Expression and Isolation of the Recombinant Silicatein-Silk Fibroin Fusion Protein

The preparation of the recombinant silicatein-silk fibroin fusion protein is preferentially performed in E. coli. The preparation of the recombinant protein in yeast and mammalian cells is also possible and has been successfully performed. The cDNA is cloned into a suitable vector, e.g. pTrcHis2-TOPO (Invitrogen). After transformation of E. coli expression of the silicatein-silk fibroin fusion protein is induced by IPTG (isopropyl-β-D-thiogalactopyranoside) (Ausubel et al. (1995) Current Protocols in Molecular Biology. John Wiley and Sons, New York). Expression of the silicatein-silk fibroin fusion protein and purification of the recombinant protein can be performed, e.g. using the histidine-tag present in the recombinant protein, on suitable affinity matrices, e.g. a Ni-NTA matrix (Skorokhod et al. (1997) Cell Mol Biol 43:509-519).

Two alternative constructs are used for the expression of the silicatein-α silk fibroin fusion protein.

1. Preparation of Fusion Protein without Protease Cleavage Site

To prepare fusion proteins with the silicatein-α polypeptide and silk fibroin a suitable expression vector (for example, pTrcHis2-TOPO vector; Invitrogen) is used. The silicatein-a cDNA—with e.g. a NcoI restriction site both at the 5′-terminus and at the 3′-terminus—is prepared. The stop codon in the silicatein-α cDNA is removed. The PCR-Technik used and for amplification, primers are used, which have the respective restriction sites. The cDNA coding for the second protein is prepared accordingly, whereby at the 5′-terminus, the same restriction site is used as at the 3′-terminus of the silicatein-α cDNA (in the example: NcoI) and at the 3′-terminus, also a NcoI restriction site.

Both cDNAs are ligated following standard procedures, purified and ligated into the pTrcHis2-TOPO vector. Ligation is performed close to a histidine-tag. Expression and purification of the fusion protein using e.g. the histidine-tag, which is present in the recombinant protein, can be performed using suitable affinity matrices, e.g. a Ni-NTA matrix (Skorokhod et al. (1997) Cell Mol Biol 43:509-519).

### 2. Separate Expression (Protease Cleavage Site)

Alternatively to the procedure under 1, a protease cleavage site (e.g. an enterokinase site) can be cloned between the cDNA coding for the silicatein-α polypeptide and the cDNA coding for a silk fibroin. After expression and purification, the fusion protein is proteolytically cleaved. Then both proteins are separated.

In the following example, expression of the fusion protein comprising the silicatein-α gene from S. domuncula and the silk fibroin gene from Mytilus galloprovincialis in E. coli is described. In this example, a silicatein insert is used, which comprises only the catalytically active domain (short silicatein form); it is also possible to use an insert which comprises the complete amino acid sequence of the protein.

The silicatein cDNAs used for the construction of cDNA encoding the fusion protein have been described.

The cDNAs coding for silicatein-α and silicatein-β from S. domuncula are; silicatein-α: AJ272013 (Krasko et al. (2000) Europ J Biochem 267:4878-4887); silicatein-β: AJ547635, AJ784227 (Schröder et al. (2004) Cell Tissue Res 316:271-280). The nucleotide sequence of S. domuncula cDNA encoding silicatein-α is shown in FIG. 3. The deduced amino acid sequence of S. domuncula cDNA encoding silicatein-α is shown in FIG. 4.

The cDNAs coding for silicatein a1-4 from Lubomirskia baicalensis are; silicatein alpha: AJ872183; silicatein a2: AJ968945; silicatein a3: AJ968946; silicatein a4: AJ968947 (Wiens et al. (2006) Dev Genes Evol 216:229-242).

The nucleotide sequence of M. galloprovincialis cDNA encoding precollagen D is shown in FIG. 5. The deduced amino acid sequence of M. galloprovincialis cDNA encoding precollagen D is shown in FIG. 6.

The nucleotide sequence and the deduced amino acid sequence of M. galloprovincialis cDNA encoding silk fibroin are shown in FIG. 7 and FIG. 8.

The vector pTrcHis2-TOPO (Invitrogen) is used for the production of the fusion protein; nucleotide sequence of pTrcHis2-TOPO, see FIG. 9. Also other expression vectors have been proven to be suitable.

The restriction enzyme NcoI (C↓CATGG) was used. This restriction enzyme has:
no restriction site in silk fibroin cDNA
no restriction site in silicatein-α cDNA
one restriction site in pTrcHis2 vector.

The following primers comprising overhangs for digestion with NcoI are used for the silicatein cDNA:

SiliNoc_For1
(SEQ ID No. 10)
CCA TGG TTC TTG TCA CAG TGG TAG TAC TG.
SiliNoc_Rev1
(SEQ ID No. 11)
CCA TGG ATA GGG TGG GAT AAG ATG CAT C.

In the first step, the sequence coding for silk fibroin is isolated from the pre-ColD cDNA using the following specific primers:

| | |
|---|---|
| Silk F | |
| 5′ GGT GGA CTC GGA GGA GC 3′. | (SEQ ID No. 12) |
| Silk HIS R | |
| 5′ ATA TCC TGG TTT GTG ATA GC 3′. | (SEQ ID No. 13) |

The silk fibroin cDNA is then cloned (T/A cloning) into the vector pTrcHis2-TOPO (Invitrogen). Bacterial strain BL21 is then transfected with the resulting plasmid.

For the silicatein-α sequence, the following reverse primer comprising an overhang is constructed, to use this overhang as a protease (enterokinase) binding site:

Sili_Endo_Rev
(SEQ ID No. 14)
5′ CTT GTC ATC GTC ATC TAG GGT GGG ATA AG 3′.

In the next step, the following primers comprising overhangs are constructed:

SiliNoc_For1
(SEQ ID No. 15)
5′ CCA TGG TTC TTG TCA CAG TGG TAG TAC TG 3′.
SiliNoc_Rev1
(SEQ ID No. 16)
5′ CCA TGG ACT TGT CAT CGT CAT CTA GG 3′.

These overhangs are used as restriction sites for the restriction enzyme NcoI. The pTrcHis2 vector also has a restriction site for this enzyme. This allows making a double digestion with the vector and with the amplified silicatein. After this digestion silicatein is cloned into pTrcHis2 vector in front of silk fibroin.

The protease binding site used to cut the silicatein from the silk fibroin (enterokinase recognition site) is shown in FIG. 10.

After digestion with NcoI the silicatein cDNA with the protease binding site is cloned into the pTrcHis2 vector in front of the silk fibroin cDNA (FIG. 11).

After transformation of E. coli expression of the silicatein-silk fibroin fusion protein is usually induced by IPTG and performed for 4 or 6 hours at 37° C. (Ausubel et al. (1995) Current Protocols in Molecular Biology. John Wiley and Sons, New York). The resulting fusion protein is purified e.g. by affinity chromatography on a Ni-NTA matrix. To separate the silicatein from silk fibroin the fusion protein is cleaved with enterokinase. The protein is then subjected to gel electrophoresis in the presence of 2-mercaptoethanol. Gel electrophoresis can be performed in 10% polyacrylamide gele with 0.1% NaDodSO₄ (polyacrylamide gel electrophoresis; PAGE). The gel is stained with Coomassie brillant blue. After cleavage, purification and subsequent PAGE the short form of the recombinant silicatein protein and silk fibroin are obtained.

### Isolation and Purification of the Silicatein-Silk Fibroin Fusion Protein Using Antibodies

The silicatein-α-silk fibroin fusion protein can be further purified on an affinity matrix. The affinitity matrix can be prepared, for example, by immobilization of a silicatein-α-specific antibody on a solid phase (CNBr-activated Sepharose or another suitable carrier). Monoclonal or polyclonal antibodies against silicatein-α can be used, which are prepared following standard methods (Osterman (1984) Methods of Protein and Nucleic Acid Research Vol. 2; Springer-Verlag [Berlin]). Coupling of the antibody to the matrix is performed according to the instructions of the manufacturer (Pharmacia). Elution of the pure silicatein-α-silk fibroin fusion protein is performed by a pH change or change in ionic strength.

Also other affinity matrices can be used.

### Detection of Silicatein Activity and Synthesis of Silica

To determine the enzyme activity of recombinant silicatein-silk fibroin fusion protein an assay is used, which is based on measurement of polymerized and precipitated silica after hydrolysis and subsequent polymerization of tetraethoxy silane (TEOS).

Measurement of enzymatic activity of recombinant silicatein is usually performed as follows. The fusion protein is dialyzed overnight against a buffer suitable for the enzymatic reaction, such as 50 mM MOPS, pH 6.8 [other buffers within a pH range of 4.5 to 10.5 are suitable too].

Fusion protein (1-50 μg) is dissolved in 1 ml of a suitable buffer, such as 50 mM MOPS (pH 6.8) and supplemented with 1 ml of 1-4.5 mM TEOS solution. Enzymatic reaction can be performed at room temperature. After an incubation period of 60 min typically 200 nmol of amorphous silica per 100 μg of silicatein are synthesized. The silica product is collected by centrifugation (12 000×g; 15 min; +4° C.), washed with ethanol and air-dried. The pellet is then hydrolyzed in 1 M NaOH. The dissolved silicate is then quantitatively determined using a molybdate-based assay, e.g. the Silicon Assay (Merck).

The following substrates can be used: tetraalkoxysilanes, trialkoxysilanols, dialkoxysilanediols, monoalkoxysilanetriols, dialkoxysilanols, monoalkoxysilanediols, monoalkoxysilanols, alkyl-, aryl- or metallotrialkoxysilanes, alkyl-, aryl- or metallosilanols, alkyl-, aryl- or metallosilanediols, alkyl-, aryl- or metallosilanetriols, alkyl-, aryl- or metallomonoalkoxysilanediols, alkyl-, aryl- or metallodialkoxysilanols, or other metal oxide precursors (alkoxy compounds of gallium, zirconium or titanium). Also mixtures of these substrates are used by the enzyme. Thus mixed polymers can also be produced.

The reaction can be performed under mild conditions. Therefore this invention contributes to the introduction of energy saving and environmentally friendly procedures.

Another reason to use silica instead of hydroxyapatite which would be a genuine regeneration is because silica is more acid resistant and hydroxyapatite would be as caries-susceptible as ‘natural’ enamel.

### Combination of Silicatein-Adhesive Protein Fusion Proteins

The silicatein silk fibroin fusion proteins and the (bio)silica produced by the silicatein can be used in combination with:

a) Human enamel-derived peptides. These peptides allow the design of silica/peptide-based nanocomposites. They are biocompatible without leaching of potentially side-effects causing resinous monomers or additives.
b) DOPA-containing polypeptides/proteins. These polypeptides/proteins can be prepared using sponge tyrosinase. They are used as glue for surface binding of silicatein/biosilica.
Combination of (Bio)Silica with Enamel-Derived Peptides/Proteins

The ratio to use peptides derived from enamel is because they deliver cohesion in enamel and will generate the best possible cohesion within the silica composite and adherence of the silica-based ‘nanocomposite’ to enamel margins of caries caused cavities. Downside of resin adhesives, they are ‘water-sensible’ and prone to hydrolytic degradation.

Combination of (Bio)Silica with Dopa-Containing Proteins and Peptides

The recombinant tyrosinase cloned from a marine sponge (Müller et al. (2004) Micron 35:87) is used to synthesize adhesive polypeptides or proteins containing 3,4-dihydroxy phenylalanine (DOPA) units. The enzymatic hydroxylation of the used tyrosine-containing proteins and peptides is performed following described procedures (Marumo and Waite (1986) Biochim Biophys Acta 872:98; Akemi Ooka and Garrell (2000) Biopolymers 57:92). One problem in the enzymatic hydroxylation of tyrosine residues within proteins or peptides to DOPA may occur by tyrosinase-mediated oxidation of the DOPA formed after tyrosine hydroxylation to further products, in particular dopaquinone. Dopaquinone is able to form cross-links with lysine residues. To reduce the formation of oxidation products from DOPA, ascorbic acid is added during hydroxylation; ascorbic acid reduces the formed dopaquinone and further oxidation products to catechol. The enzyme is removed by centrifugation/filtration (micropore filter) during hydroxylation of low-molecular-weight peptides. The ascorbic acid is separated from the products by reverse-phase HPLC.

The recombinant sponge tyrosinase is prepared using the “GST Fusions” system (Amersham). The resulting GST fusion protein is purified by affinity chromatography on glutathione-Sepharose 4B. To separate the glutathione-5-transferase from the recombinant sponge enzymes, the fusion protein is cleaved with thrombin. The determination of the enzymatic activity of the tyrosinase is performed in a phosphate buffer using L-tyrosine as substrate. The conversion of tyrosine in L-DOPA (3,4-dihydroxy phenylalanine) is measured at 280 nm.

Also silicatein can be modified using recombinant sponge tyrosinase which converts monophenols (e.g. tyrosine residues) in diphenols.

The resulting glue proteins can be used to link building blocks made of biosilica (synthesized by silicatein) under formation of higher-ordered structures.

### Application of the Silicatein-α Silk Fibroin Fusion Proteins

Further aspects of the invention are the following applications of the silicatein-α silk fibroin fusion proteins.

1.) Application for surface modification of dental materials (improvement of biocompatibility, higher stability and higher porosity compared with the presently used materials).
2.) Application for the preparation of novel dental materials (composite materials) such as dental replacement materials.
3.) Application for the preparation of coatings for dental materials made of metals, metal oxides, plastics and other materials; in particular preparation of monomolecular layers on these materials (biologisation; formation of a stable connection between bone and dental materials).
4.) Use of the adhesive proteins (“underwater glues”) present in the fusion protein for the attachment of silicatein and biosilica building blocks (biosilica particles synthesized by silicatein) onto surfaces (metal, glass etc.) and to link biosilica building blocks under formation of higher-ordered structures.
5.) Use of biosilica as filler particles in dentistry. Biosilica has two advantages compared to conventional materials: 1. it is synthesized environmentally friendly, because it can be generated enzymatically using recombinant enzymes (silicateins) or bioreactors (primmorphs). Surface modification could be done with silicase. There is no need for high energy or aggressive chemicals.

### The Legends to the Figures and Sequence Protocols are as Follows.

FIG. 1. First description of the biochemical adhesive mechanism in Holothuria. Left: Two Cuvier tubuli. A, inner cylinder; B, outer cylinder. Right: Cuvier tubulus adhering to paraffin (Müller and Zahn (1972) Cytobiologie 3: 335-351).

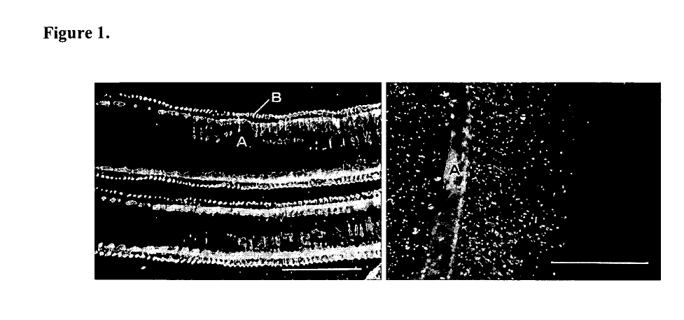

FIG. 2. Conversion of monophenols in diphenols (and further oxidation to quinones) by sponge tyrosinase.

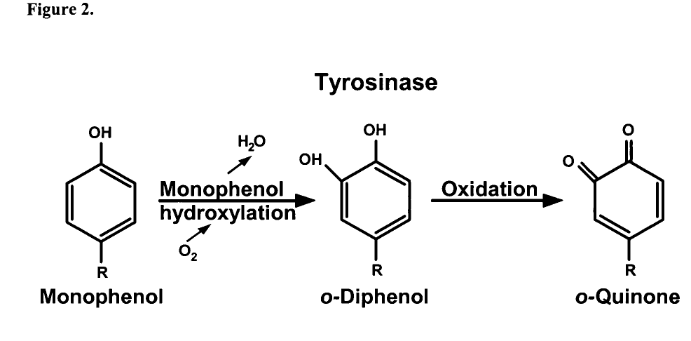

FIG. 3. Nucleotide sequence of Suberites domuncula cDNA encoding silicatein-α. The sequence used for the construction of primers are underlined and labelled in bold letters. The ATG start codon is double-underlined (SEQ ID No. 5).

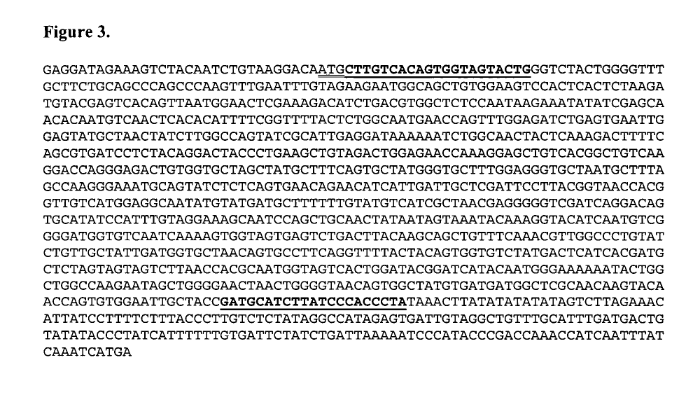

FIG. 4. Deduced amino acid sequence of Suberites domuncula cDNA encoding silicatein-α.

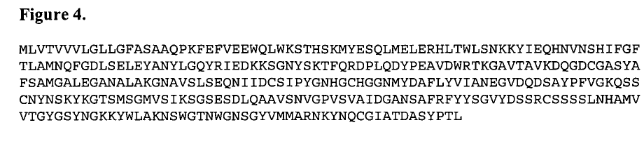

FIG. 5. Nucleotide sequence of Mytilus galloprovincialis cDNA encoding precollagen D. The sequence used for the construction of primers are underlined and labelled in bold letters (SEQ ID No. 6).

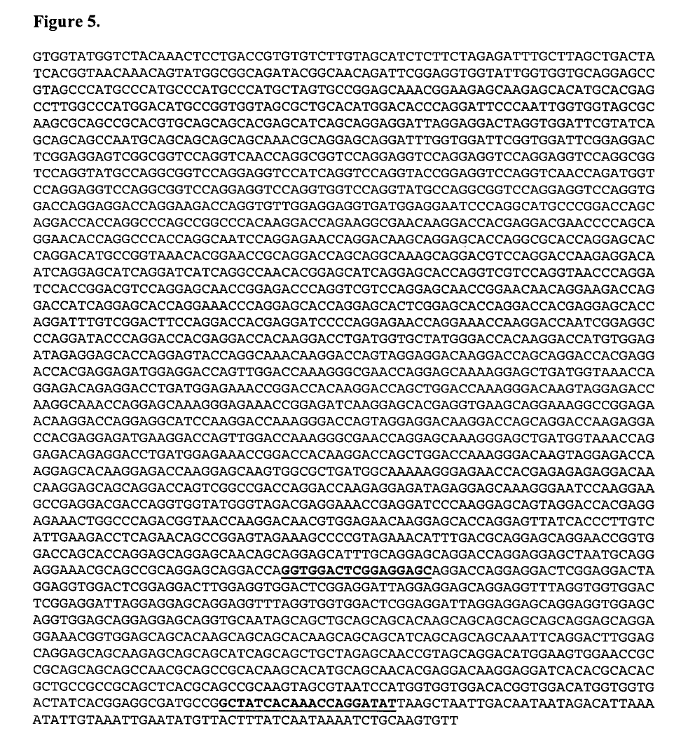

FIG. 6. Deduced amino acid sequence of Mytilus galloprovincialis cDNA encoding precollagen D (SEQ ID No. 7).

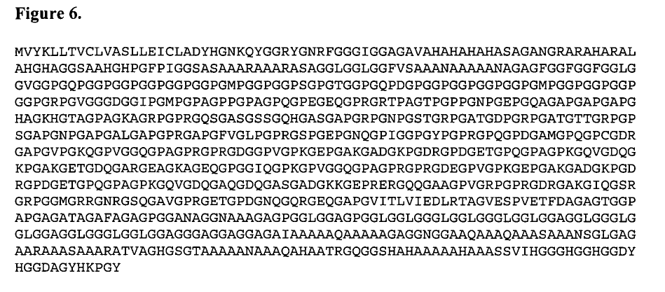

FIG. 7. Deduced amino acid sequence of Mytilus galloprovincialis cDNA encoding silk fibroin (SEQ ID No. 8).

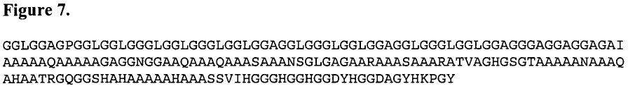

FIG. 8. Nucleotide sequence and deduced amino acid sequence of Mytilus galloprovincialis cDNA encoding silk fibroin. The sequence used for the construction of the forward and reverse primers are underlined and labelled in bold letters (SEQ ID No. 9).

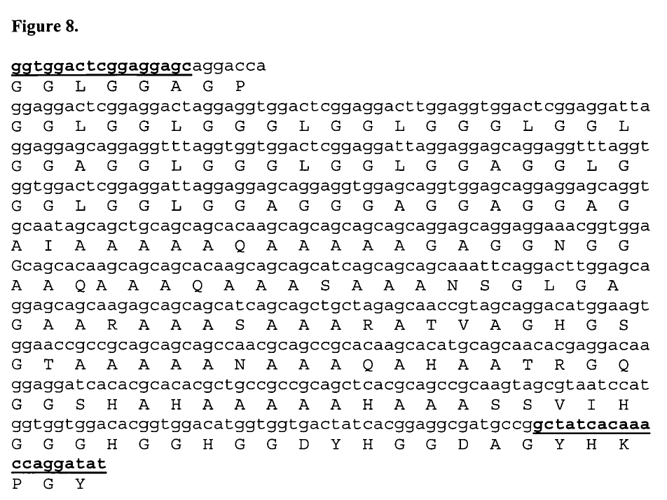

FIG. 9. Nucleotide sequence of vector pTrcHis2-TOPO (Invitrogen).

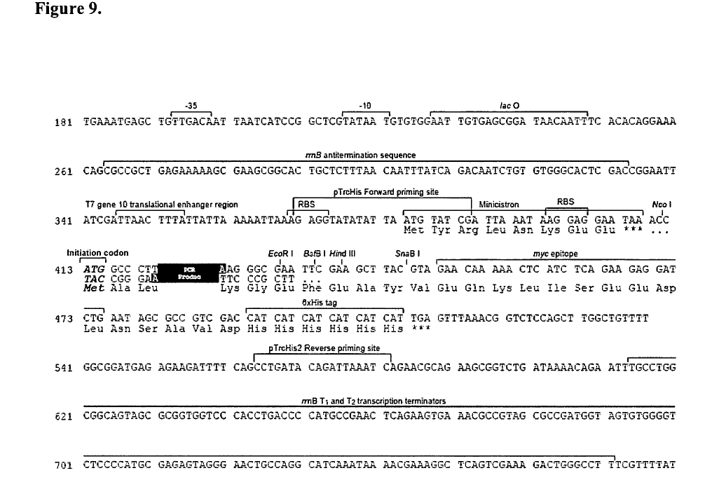

FIG. 10. Protease binding site (enterokinase recognition site) used to cut silicatein from silk fibroin by enterokinase.

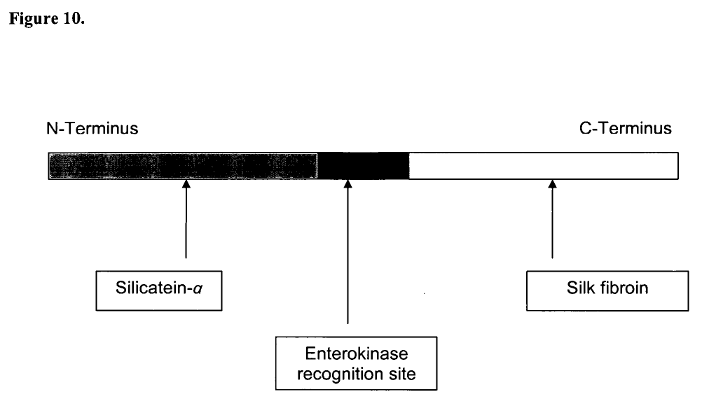

FIG. 11. Cloning of the silicatein gene with the protease binding site into the pTrcHis2 vector in front of the silk fibroin cDNA after the digestion with NcoI.

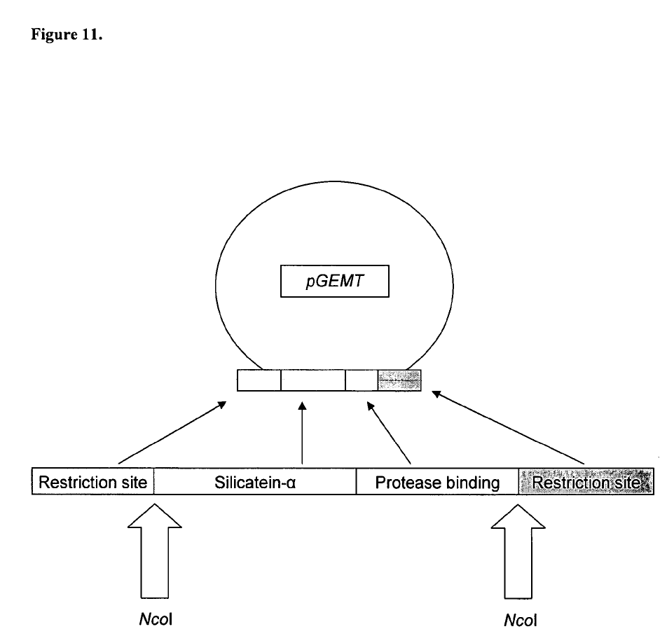

SEQ ID No. 1: Amino acid sequence of the silicatein-α polypeptide from Suberites domuncula (rSILICAα_SUBDO),

SEQ ID No. 2: Amino acid sequence of the silk fibroin polypeptide from Mytilus galloprovincialis (rSILKFIB_MYTGA),

SEQ ID No. 3: Nucleic acid sequence of the cDNA coding for the silicatein-α polypeptide from Suberites domuncula.

SEQ ID No. 4: Nucleic acid sequence of the cDNA coding for the silk fibroin polypeptide from Mytilus galloprovincialis.

1-39. (canceled)

40. A fusion protein, comprising a silica forming enzyme and an adhesive protein.

41. The fusion protein according to claim 40 containing a protease cleavage site between the silica forming enzyme and the adhesive protein.

42. The fusion protein according to claim 40, wherein the silica forming enzyme is a silicatein.

43. The fusion protein according to claim 40, wherein the silica forming enzyme is silicatein-α.

44. The fusion protein according to claim 40, wherein the adhesive protein is silk fibroin.

45. The fusion protein according to claim 41, wherein the protease cleavage site is an enterokinase cleavage site.

46. A nucleic acid encoding a fusion protein according to claim 40.

47. A nanocomposite material comprising a fusion protein according to claim 40.

48. The nanocomposite material according to claim 47, together with suitable additives and supplements.

49. The nanocomposite material according to claim 47, wherein one or several components is/are present in the form of a depot compound or as a precursor together with a suitable dilution solution or a carrier substance.

50. The nanocomposite material according to claim 47, wherein the composition modulates calcium phosphate precipitation, enamel cell recruitment and/or exhibits antibacterial activity.

51. A method for in vitro or non-human in vivo synthesis of silicon dioxide, silicones and/or other metal oxides, as well as mixed polymers wherein said method comprises the use of a fusion protein of claim 40.

52. The method according to claim 51, wherein the fusion protein comprises an adhesive protein domain, which exhibits at least 25% sequence similarity to the sequence shown in SEQ ID No. 1 and/or a silicatein domain, which exhibits at least 25% sequence similarity to the sequence shown in SEQ ID No. 1.

53. The method according to claim 51, wherein silicic acid; silicates, monoalkoxysilanetriols; monoalkoxysilanediols; monoalkoxysilanols: dialkoxysilane-diols; dialkoxysilanols; trialkoxysilanols; tetraalkoxysilanes; alkyl-, aryl- or metallo-silanetriols; alkyl-, aryl- or metallo-silanediols; alkyl-, aryl- or metallo-silanols; alkyl-, aryl- or metallo-monoalkoxysilanediols; alkyl-, aryl- or metallo-monoalkoxysilanols-alkyl-, aryl- or metallo-dialkoxysilanols; alkyl-, aryl- or metallo-trialkoxysilanes; or other metal oxide precursor compounds are used as substrates for synthesis.

54. The method according to claim 53, wherein mixed polymers of defined composition are produced using defined mixtures of compounds.

55. The method according to claim 51, wherein defined 2- and 3-dimensional structures are produced by surface binding of the fusion protein on glass, metals, metal oxides, plastics, or biopolymers used as a template.

56. A method for the preparation of DOPA-containing proteins and peptides using recombinant sponge tyrosinase.

57. A method for binding silicatein/biosilica building blocks on surfaces wherein the method uses DOPA-containing polypeptides.

58. The nucleic acid according to claim 46, which encodes (a) a fusion protein (chimeric protein) construct, or (b) a construct with separate protein expression (protease cleavage site).

59. The nucleic acid according to claim 46, wherein the nucleic acid comprises at least one intron and/or a polyA sequence.

60. A vector comprising a nucleic acid according to claim 46.

61. A vector comprising a polypeptide according to claim 40.

62. A host cell transfected with a vector according to claim 65.

63. The polypeptide according to claim 40, wherein the polypeptide is present in a prokaryotic or eukaryotic cell extract or lysate.

64. The polypeptide according to claim 40, wherein the polypeptide is purified and essentially free from other proteins.

65. A method for modulating the resorption of silicones and silicon monomers in silicone implants wherein the method comprises the use of a polypeptide of claim 40 or a nucleic acid encoding the polypeptide.

66. A method for coating of metals, metal oxides, plastics and other materials wherein said method uses a fusion protein of claim 40.

67. A composition of matter comprising silicatein-silk fibroin fusion proteins with biocompatible, human enamel-derived peptides/proteins (silica/peptide-based nanocomposites).

68. A composition of matter comprising silicatein-silk fibroin fusion proteins with DOPA-containing proteins and peptides.
